(19) United States
(12) Patent Application Publication
(10) Pub. No.: US 2021/0315834 A1
(43) Pub. Date: Oct. 14, 2021

(54) TREATMENT OF ALOPECIA

(71) Applicant: Sol-Gel Technologies Ltd., Ness Ziona (IL)

(72) Inventors: Ori Nov, Tarum (IL); Marcel Zighelboim, Kiryat Motzkin (IL); Karine Neimann, Ness Ziona (IL)

(73) Assignee: Sol-Gel Technologies Ltd., Ness Ziona (IL)

(21) Appl. No.: 17/227,459

(22) Filed: Apr. 12, 2021

Related U.S. Application Data
(60) Provisional application No. 63/008,745, filed on Apr. 12, 2020.

(51) Int. Cl. A61K 31/05; A61K 45/06; A61K 9/00; A61K 31/517; A61K 31/4174; A61K 9/06

(52) U.S. Cl. CPC A61K 31/05; A61K 45/06; A61K 9/06; A61K 31/517; A61K 31/4174; A61K 9/0014

(57) ABSTRACT

The present invention is directed to a composition comprising aryl hydrocarbon receptor (AhR) agonists and combinations for the prevention and treatment of alopecia and hair loss, e.g. when such conditions are caused by chemotherapy, radiation therapy or hormone replacement therapy.

### CROSS REFERENCE TO RELATED APPLICATIONS

This application claims the benefit of U.S. Ser. No. 63/008,745, filed on Apr. 12, 2020 (and entitled Treatment of Alopecia), which is incorporated in their entirety herein by reference.

### FIELD OF THE INVENTION

The present disclosure relates to the prevention and treatment of alopecia and hair loss, e.g. when such conditions are caused by chemotherapy, radiation therapy or hormone replacement therapy.

### BACKGROUND OF THE INVENTION

Chemotherapy-induced alopecia (CIA) is an acquired form of hair loss that affects patient quality of life, negatively impacts body image, sexuality, and self-esteem, and provides a strong indication of the individual's health status, with most people associating it with cancer.

The prevention models of CIA include pharmacological and physical interventions. The biological intervention is given through antibodies neutralize doxorubicin activity, vasoconstrictors preventing drug entry to the HF, antioxidants, immunosuppressive and inhibitor of apoptosis like caspase-3. The non-drug therapy includes sowing blood flew by occlusion or scalp cooling.

Usage of biotin for hair loss is also known. Biotin is a water-soluble vitamin that's a part of the vitamin B family. It's also known as vitamin H. The human body needs biotin to help convert certain nutrients into energy, and plays an important role in the health condition of hair, skin, and nails. Biotin-deficiency may cause hair loss or a scaly red rash. However, a deficiency is rare. In most cases, the biotin supplemented by diet is satisfactory. Still, many people are increasing their intake in hopes of additional benefits.

The aryl hydrocarbon receptor (AhR) is a ligand-activated transcription factor that is activated by small molecules provided by the diet, microorganisms, metabolism and pollutants. The AhR is expressed in keratinocytes and specifically in hair follicles. The AhR was found to play an intrinsic role in survival and proliferation of immune cells and is essential for murine skin barrier integrity of the epidermis (J. Invest Dermatol. 2016 November; 136(11):2260-2269). When inactive, AhR is localized in the cytoplasm as part of a complex formed by a dimer of the 90-kDa heat shock protein (HSP90). HSP90 stabilizes AhR in a conformation of high affinity for its ligands. Ligand binding releases AhR-interacting protein from the complex and triggers conformational changes in AhR that expose its nuclear localization signal, leading to AhR translocation to the nucleus. Once in the nucleus, the association of AhR with the AhR nuclear translocator (ARNT) results in the transcriptional control of multiple target genes (Cell Stress Chaperones. 2004 March; 9(1): 4-20). These genes include several xenobiotic metabolizing enzymes including the microsomal cytochrome P450-dependent monooxygenases including cytochrome P450 family-1 subfamily-A polypeptide-1 (CYP1A1), cytochrome P450 family-1 subfamily-A Polypeptide-2 (CYP1A2), cytochrome P450 family-1 subfamily-B polypeptide-1 (CYP1B1) and NAD(P)H-quinone oxidoreductase. This family is accompanied with anti-apoptosis activity like carcinogenesis, and when CYP1 A1 inhibited it modulates p53-mediated apoptosis (BMC Cancer. 2009; 9: 187). Cancer treatment with chemotherapeutic agents is associated with severe side effects due to the occurrence of apoptosis in several sensitive tissues (such as the hematopoietic system or epithelia of digestive tract) as a result of drug cytotoxicity. This apoptosis largely depends on p53, a key mediator of cellular mechanism of stress response.

Since chemotherapy drugs cause death to cells that divide rapidly, organs such as hair follicle (HF) and matrix keratinocytes affected and damaged. Chemotherapy induces melanin clumping and incontinence, down-regulates proliferation and massively up-regulates apoptosis of (predominantly) hair matrix keratinocytes, prematurely induces catagen accompanied with up-regulation of p53. The chemotherapy-induced hair follicle apoptosis is mainly mediated by p53, and a prevention of activating this molecule and therefore preventing chemotherapy induced hair loss. Chemotherapy drugs such as pyrimidine analogs and topoisomerase II inhibitors induce cellular DNA damage response and inevitably activate p53.

Many papers described the negative correlation between higher AhR activity and reduction p53 can lead to antiapoptotic activity (J Biol Chem. 2004 Jun. 25; 279(26):27187-93; Int J Clin Exp Pathol. 2014 Oct. 15; 7(11):7931-7). Tapinarof is a small molecule which is agonizing AhR to induce production of CYP1A1, CYP1A2 and CYP1B1. Upregulation of AhR-dependent cytochromes will probably lead to reduction in p53 activity and to anti-apoptotic activity of the hair follicle.

### SUMMARY OF THE INVENTION

The present invention provides a method of treating, preventing or alleviating chemotherapy-induced, radiation-therapy-induced, or hormone-replacement-therapy-induced alopecia or hair loss in a subject in need thereof, wherein the method comprises administering to the subject a therapeutically effective amount of a topical pharmaceutical composition comprising at least one aryl hydrocarbon receptor (AhR) agonist in a concentration of between 0.01% to about 5% w/w, and a pharmaceutically acceptable carrier or excipient.

In other embodiments, the topical composition comprises an AhR agonist alone or in combination with other active agents, such as topical EGFR inhibitors or topical vasoconstrictors. The active agents in the topical pharmaceutical composition of this invention may be in an encapsulated or non-encapsulated form, according to pharmaceutical or formulation needs. The above compositions are useful for the treatment, prevention or alleviation or treatment of chemotherapy-induced, radiation-induced or hormone-replacement-therapy-induced alopecia (hair loss).

### DETAILED DISCUSSION OF THE INVENTION

The present disclosure relates to composition and methods for preventing hair loss, slowing hair loss and/or treating hair loss.

The present invention relates to use of aryl hydrocarbon receptor (AhR) agonists (such as tapinarof) alone or in combination with EGFR inhibitors and/or with vasoconstrictors, for treating chemotherapy, radiation or hormone-replacement-induced alopecia. The present invention provides a topical composition comprising an AhR agonist alone or in combination with other active agents, such as EGFR inhibitors or vasoconstrictors. The Active agents involved in treatment can be, each one independently, or in combination, formulated differently, e.g. as a cream or suppositories, and administered independently, or in combination, before, during or after the chemo/radiation/hormone-replacement therapy protocol.

The present invention provides, in an aspect, a topical pharmaceutical composition for use in treating, preventing or alleviating of chemotherapy-induced, radiation-induced or hormone-replacement-therapy-induced alopecia or hair loss in a subject in need thereof, the topical pharmaceutical composition comprising at least one aryl hydrocarbon receptor (AhR) agonist in a concentration of between 0.01% to about 5% w/w, from 0.01% to about 1% w/w, from about 1% to about 3% w/w, or from about 3% to about 5% w/w, and a pharmaceutically acceptable carrier or excipient.

In certain embodiments, the present invention provides a topical pharmaceutical composition for use in treating, preventing or alleviating of chemotherapy-induced alopecia or hair loss.

In certain embodiments, the present invention provides a topical pharmaceutical composition for use in treating, preventing or alleviating of radiation-induced alopecia or hair loss.

In certain embodiments, the present invention provides a topical pharmaceutical composition for use in treating, preventing or alleviating of hormone-replacement-therapy-induced alopecia or hair loss.

In certain embodiments, the topical pharmaceutical composition comprises an AhR agonist in a concentration of between 0.01% to about 1% w/w. In certain embodiments, the topical pharmaceutical composition comprises an AhR agonist in a concentration of between 0.01% to about 0.1% w/w. In certain embodiments, the topical pharmaceutical composition comprises an AhR agonist in a concentration of between 0.1% to about 1% w/w.

In certain embodiments, the topical pharmaceutical composition comprises an AhR agonist in a concentration of between 1% to about 3% w/w. In certain embodiments, the topical pharmaceutical composition comprises an AhR agonist in a concentration of between 1% to about 2% w/w. In certain embodiments, the topical pharmaceutical composition comprises an AhR agonist in a concentration of between 2% to about 3% w/w.

In certain embodiments, the topical pharmaceutical composition comprises an AhR agonist in a concentration of between 3% to about 5% w/w. In certain embodiments, the topical pharmaceutical composition comprises an AhR agonist in a concentration of between 3% to about 4% w/w. In certain embodiments, the topical pharmaceutical composition comprises an AhR agonist in a concentration of between 4% to about 5% w/w.

In certain embodiments, the AhR agonist is selected from the group consisting of tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, Norisoboldine and any combination thereof.

In certain embodiments, the AhR agonist is tapinarof. In certain embodiments, the AhR agonist is FICZ. In certain embodiments, the AhR agonist is Indirubin. In certain embodiments, the AhR agonist is ITE. In certain embodiments, the AhR agonist is L-Kynurenine. In certain embodiments, the AhR agonist is a Flavonoid. In certain embodiments, the AhR agonist is Leflunomide. In certain embodiments, the AhR agonist is Norisoboldine.

In certain embodiments, the AhR agonist is a combination of AhR agonists.

In certain embodiments, the topical pharmaceutical composition further comprises at least one EGFR inhibitor in a concentration of from about 0.01% to about 10%, from 0.01% to about 1% w/w, from about 1% to about 3% w/w, from about 3% to about 5% w/w or from about 5% to about 10% w/w.

In certain embodiments, the topical pharmaceutical composition comprises an EGFR inhibitor in a concentration of between 0.01% to about 1% w/w. In certain embodiments, the topical pharmaceutical composition comprises an EGFR inhibitor in a concentration of between 0.01% to about 0.1% w/w. In certain embodiments, the topical pharmaceutical composition comprises an EGFR inhibitor in a concentration of between 0.1% to about 1% w/w.

In certain embodiments, the topical pharmaceutical composition comprises an EGFR inhibitor in a concentration of between 1% to about 3% w/w. In certain embodiments, the topical pharmaceutical composition comprises an EGFR inhibitor in a concentration of between 1% to about 2% w/w. In certain embodiments, the topical pharmaceutical composition comprises an EGFR inhibitor in a concentration of between 2% to about 3% w/w.

In certain embodiments, the topical pharmaceutical composition comprises an EGFR inhibitor in a concentration of between 3% to about 5% w/w. In certain embodiments, the topical pharmaceutical composition comprises an EGFR inhibitor in a concentration of between 3% to about 4% w/w. In certain embodiments, the topical pharmaceutical composition comprises an EGFR inhibitor in a concentration of between 4% to about 5% w/w.

In certain embodiments, the topical pharmaceutical composition comprises an EGFR inhibitor in a concentration of between 5% to about 10% w/w. In certain embodiments, the topical pharmaceutical composition comprises an EGFR inhibitor in a concentration of between 5% to about 7% w/w. In certain embodiments, the topical pharmaceutical composition comprises an EGFR inhibitor in a concentration of between 7% to about 10% w/w.

In certain embodiments, the topical pharmaceutical composition further comprises at least one vasoconstrictor compound in a concentration of between about 0.01% to about 5% w/w.

Usage of vasoconstrictors for chemo-related hair loss is known for several years (Int J Cancer. 2015 Jan. 1; 136(1): 195-203), but here it can be even better: tapinarof doesn't need to be delivered through the blood system (as in systemic administration), since it can penetrate into the hair follicle directly (as in targeted, or topical, administration), and the vasoconstrictor will prevent the chemotherapy to reach the hair follicle (HF). In addition, since tapinarof is a positive regulator of Cytochromes P450 (CYPs) enzymes, it may be beneficial to prevent it from reaching to other, un-related organs.

In certain embodiments, the topical pharmaceutical composition comprises a vasoconstrictor compound in a concentration of between 0.01% to about 1% w/w. In certain embodiments, the topical pharmaceutical composition comprises a vasoconstrictor compound in a concentration of between 0.01% to about 0.1% w/w. In certain embodiments, the topical pharmaceutical composition comprises a vasoconstrictor compound in a concentration of between 0.1% to about 1% w/w.

In certain embodiments, the topical pharmaceutical composition comprises a vasoconstrictor compound in a concentration of between 1% to about 3% w/w. In certain embodiments, the topical pharmaceutical composition comprises a vasoconstrictor compound in a concentration of between 1% to about 2% w/w. In certain embodiments, the topical pharmaceutical composition comprises a vasoconstrictor compound in a concentration of between 2% to about 3% w/w.

In certain embodiments, the topical pharmaceutical composition comprises a vasoconstrictor compound in a concentration of between 3% to about 5% w/w. In certain embodiments, the topical pharmaceutical composition comprises a vasoconstrictor compound in a concentration of between 3% to about 4% w/w. In certain embodiments, the topical pharmaceutical composition comprises a vasoconstrictor compound in a concentration of between 4% to about 5% w/w.

In certain embodiments, the topical pharmaceutical composition further comprises (i) at least one EGFR inhibitor in a concentration of from about 0.01% to about 10% w/w; and (ii) at least one vasoconstrictor compound in a concentration of between about 0.01% to about 5% w/w.

In certain embodiments, the EGFR inhibitor is selected from the group consisting of erlotinib, gefitinib, lapatinib, cetuximab, panitumumab, vandetanib, necitumumab, osimertinib, and combinations thereof.

In certain embodiments, the EGFR inhibitor is erlotinib. In certain embodiments, the EGFR inhibitor is gefitinib. In certain embodiments, the EGFR inhibitor is lapatinib. In certain embodiments, the EGFR inhibitor is cetuximab. In certain embodiments, the EGFR inhibitor is panitumumab. In certain embodiments, the EGFR inhibitor is vandetanib. In certain embodiments, the EGFR inhibitor is necitumumab. In certain embodiments, the EGFR inhibitor is osimertinib.

In certain embodiments, the vasoconstrictor compound is selected from the group consisting of oxymetazoline, xylometazoline, brimonidine, a vasopressin analog, epinephrine, norepinephrine, phenylephrine (Sudafed PE), dopamine, dobutamine, a serotonin 5-hydroxytryptamine agonist (a triptan) and any combination thereof.

In certain embodiments, the vasoconstrictor compound is oxymetazoline. In certain embodiments, the vasoconstrictor compound is xylometazoline. In certain embodiments, the vasoconstrictor compound is brimonidine. In certain embodiments, the vasoconstrictor compound is a vasopressin analog. In certain embodiments, the vasoconstrictor compound is epinephrine. In certain embodiments, the vasoconstrictor compound is norepinephrine. In certain embodiments, the vasoconstrictor compound is phenylephrine. In certain embodiments, the vasoconstrictor compound is dopamine. In certain embodiments, the vasoconstrictor compound is dobutamine. In certain embodiments, the vasoconstrictor compound is a triptan.

In certain embodiments, the active agents in the topical pharmaceutical composition of this invention may be in an encapsulated or non-encapsulated form, according to pharmaceutical or formulation needs. In certain embodiments, encapsulation of tapinarof described in WO 2021/059281 (to Sol-Gel Technologies) which is incorporated herein by reference.

In certain embodiments, the topical pharmaceutical composition is formulated as cream, an ointment, a gel, a lotion, a spray, a shampoo, a patch, or a foam. In certain embodiments, the topical pharmaceutical composition is formulated as a cream. In certain embodiments, the topical pharmaceutical composition is formulated as an ointment. In certain embodiments, the topical pharmaceutical composition is formulated as a gel. In certain embodiments, the topical pharmaceutical composition is formulated as a lotion. In certain embodiments, the topical pharmaceutical composition is formulated as a spray. In certain embodiments, the topical pharmaceutical composition is formulated as a shampoo. In certain embodiments, the composition is formulated as a patch. In certain embodiments, the topical pharmaceutical composition is formulated as a foam.

The present invention further provides, a method of treating, preventing or alleviating of chemotherapy-induced, radiation-therapy-induced, or hormone-replacement-therapy-induced alopecia or hair loss in a subject in need thereof, the method comprising the step of administering to the subject a therapeutically effective amount of the topical pharmaceutical composition described above.

In certain embodiments, the present invention further provides a method of preventing or alleviating of chemotherapy-induced alopecia or hair loss by administering topical AhR agonist alone or in combination with other active agents, such as topical EGFR inhibitors or topical vasoconstrictors.

In certain embodiments, the present invention further provides a method of preventing or alleviating of radiation-therapy-induced alopecia or hair loss by administering topical AhR agonist alone or in combination with other active agents, such as topical EGFR inhibitors or topical vasoconstrictors.

In certain embodiments, the present invention further provides a method of preventing or alleviating of hormone-replacement-therapy-induced alopecia or hair loss by administering topical at least one AhR agonist alone or in combination with other active agents, such as topical EGFR inhibitors or topical vasoconstrictors. In another embodiment, the at least one AhR agonist is selected from group consisting of tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, Norisoboldine and any combination thereof. In another embodiment, the AhR agonist is tapinarof. In another embodiment, the EGFR inhibitor is selected from the group consisting of erlotinib, gefitinib, lapatinib, cetuximab, panitumumab, vandetanib, necitumumab, osimertinib, and combinations thereof. In another embodiment the EGFR inhibitor is erlotinib. In another embodiment the vasoconstrictor compound is selected from the group consisting of oxymetazoline, xylometazoline, brimonidine, a vasopressin analog, epinephrine, norepinephrine, phenylephrine (Sudafed PE), dopamine, dobutamine, a serotonin 5-hydroxytryptamine agonist (a triptan) and any combination thereof. In another embodiment, the vasoconstrictor compound is oxymetazoline.

In certain embodiments, the method comprising the step of administering to the subject a therapeutically effective amount of the topical pharmaceutical composition comprising an AhR agonist. In another embodiment the AhR agonist is tapinarof.

In certain embodiments, the method comprising the step of administering to the subject a therapeutically effective amount of the topical pharmaceutical composition comprising an AhR agonist and a vasoconstrictor compound. In another embodiment, the composition comprises tapinarof and oxymetazoline.

In certain embodiments, the method comprising the step of administering to the subject a therapeutically effective amount of the topical pharmaceutical composition comprising an AhR agonist and an EGFR inhibitor. In another embodiment, the composition comprises tapinarof and erlotinib.

In certain embodiments, the method comprising the step of administering to the subject a therapeutically effective amount of the topical pharmaceutical composition comprising an AhR agonist and a vasoconstrictor and an EGFR inhibitor.

In certain embodiments, this invention provides a method of treating, preventing or alleviating of chemotherapy-induced, radiation-therapy-induced, or hormone-replacement-therapy-induced alopecia or hair loss in a subject in need thereof, the method comprising the step of administering to the subject a therapeutically effective amount of at least one aryl hydrocarbon receptor (AhR) agonist in a concentration of between 0.01% to about 1% w/w, from about 1% to about 3% w/w, or from about 3% to about 5% w/w.

In other embodiments, the method of this invention comprises administering a combination of at least one aryl hydrocarbon receptor (AhR) agonist in a concentration of between 0.01% to about 1% w/w, from about 1% to about 3% w/w, or from about 3% to about 5% w/w, in combination with at least one EGFR inhibitor in a concentration of from about 0.01% to about 1% w/w, from about 1% to about 3% w/w, from about 3% to about 5% w/w or from about 5% to about 10% w/w. In another embodiment, the administration of each is independently, or formulated as a combination topical composition. In another embodiment, the at least one hydrocarbon receptor (AhR) agonist is administered independently from the at least one EGFR inhibitor, wherein the at least one hydrocarbon receptor (AhR) agonist is formulated as a topical composition and the at least one EGFR inhibitor is formulated as a topical composition or as a systemic composition.

In other embodiments, the method of this invention comprises administering a combination of at least one aryl hydrocarbon receptor (AhR) agonist in a concentration of between 0.01% to about 1% w/w, from about 1% to about 3% w/w, or from about 3% to about 5% w/w, in combination with at least one at least one vasoconstrictor compound in a concentration of between about 0.01% to about 5% w/w. In another embodiment, the administration of each is independently, or formulated as a combination topical composition. In another embodiment, the at least one hydrocarbon receptor (AhR) agonist is administered independently from the at least one vasoconstrictor compound, wherein the at least one hydrocarbon receptor (AhR) agonist is formulated as a topical composition and the at least vasoconstrictor compound is formulated as a topical composition or as a systemic composition.

In other embodiments, the method of this invention comprises administering a combination of at least one aryl hydrocarbon receptor (AhR) agonist in a concentration of between 0.01% to about 1% w/w, from about 1% to about 3% w/w, or from about 3% to about 5% w/w, in combination with at least one EGFR inhibitor in a concentration of from about 0.01% to about 1% w/w, from about 1% to about 3% w/w, from about 3% to about 5% w/w or from about 5% to about 10% w/w and at least one vasoconstrictor compound in a concentration of between about 0.01% to about 5% w/w. In another embodiment, the administration of each is independently, or formulated as a combination topical composition. In another embodiment, the at least one hydrocarbon receptor (AhR) agonist is administered independently from the at least one EGFR inhibitor and the at least one vasoconstrictor compound, wherein the at least one hydrocarbon receptor (AhR) agonist is formulated as a topical composition and the at least one EGFR inhibitor is formulated as a topical composition or as a systemic composition, and wherein the at least one vasoconstrictor compound is formulated as a topical composition or as a systemic composition. In another embodiment, the AhR agonist is tapinarof, the EGFR inhibitor is erlotinib and the vasoconstrictor compound is oxylometazoline.

In other embodiments, the method of this invention comprises administering a topical composition comprising of at least one aryl hydrocarbon receptor (AhR) agonist in a concentration of between 0.01% to about 1% w/w, from about 1% to about 3% w/w, or from about 3% to about 5% w/w, and at least one EGFR inhibitor in a concentration of from about 0.01% to about 1% w/w, from about 1% to about 3% w/w, from about 3% to about 5% w/w or from about 5% to about 10% w/w; wherein the method further comprises administering at least one vasoconstrictor compound in a concentration of between about 0.01% to about 5% w/w wherein the at least one vasoconstrictor compound is formulated as a topical composition or as a systemic composition.

In other embodiments, the method of this invention comprises administering a topical composition comprising of at least one aryl hydrocarbon receptor (AhR) agonist in a concentration of between 0.01% to about 1% w/w, from about 1% to about 3% w/w, or from about 3% to about 5% w/w, and at least one vasoconstrictor compound in a concentration of between about 0.01% to about 5% w/w; wherein the method further comprises administering at least one EGFR inhibitor in a concentration of from about 0.01% to about 1% w/w, from about 1% to about 3% w/w, from about 3% to about 5% w/w or from about 5% to about 10% w/w, wherein the at least one EGFR inhibitor is formulated as a topical composition or as a systemic composition.

The present invention broadly relates to use of aryl hydrocarbon receptor (AhR) agonists, such as tapinarof, for treating chemotherapy-induced alopecia. Active agents involved in treatment can be, each one independently, or in combination, formulated differently, e.g. as a cream or suppositories, and administered independently, or in combination, before, during or after the chemotherapy protocol.

In certain embodiments, the composition is administered before the chemotherapy protocol. In certain embodiments, the composition is administered during the chemotherapy protocol. In certain embodiments, the composition is administered after the chemotherapy protocol.

In certain embodiments, the topical pharmaceutical composition is administered before the radiotherapy protocol. In certain embodiments, the topical pharmaceutical composition is administered during the radiotherapy protocol. In certain embodiments, the topical pharmaceutical composition is administered after the radiotherapy protocol.

In certain embodiments, the composition is administered before the hormone-replacement-therapy protocol. In certain embodiments, the composition is administered during the hormone-replacement-therapy protocol. In certain embodiments, the composition is administered after the hormone-replacement-therapy protocol.

In certain embodiments, according to the method of the present invention, the at least one AhR agonist and the at least one EGFR inhibitor exhibit an additive effect. In certain embodiments, the at least one AhR agonist and the at least one EGFR inhibitor exhibit a synergistic effect.

In certain embodiments, according to the method of the present invention, the at least one AhR agonist and the at least one vasoconstrictor compound exhibit an additive effect. In certain embodiments, the at least one AhR agonist and the at least one vasoconstrictor compound exhibit a synergistic effect.

In certain embodiments, according to the method of the present invention, the at least one AhR agonist, the at least one EGFR inhibitor and the at least one vasoconstrictor compound exhibit an additive effect. In certain embodiments, the at least one AhR agonist, the at least one EGFR inhibitor and the at least one vasoconstrictor compound exhibit a synergistic effect.

In certain embodiments, the alopecia is selected from the group consisting of alopecia areata totalis, alopecia areata universalis and androgenetic alopecia, telogen effluvium, tinea capitis, hypotrichosis, and hereditary hypotrichosis simplex. In certain embodiments, the alopecia is alopecia areata totalis. In certain embodiments, the alopecia is alopecia areata universalis and androgenetic alopecia. In certain embodiments, the alopecia is telogen effluvium. In certain embodiments, the alopecia is tinea capitis. In certain embodiments, the alopecia is hypotrichosis. In certain embodiments, the alopecia is hereditary hypotrichosis simplex.

In certain embodiments, the method comprises systemic administration of a therapeutically effective amount of the composition to the subject affected by the hair loss until the hair loss disorder is cured, prevented or alleviated or according to doctor's instructions.

In certain embodiments, the method comprises topical application of a therapeutically effective amount of the composition to the scalp or skin portion of the subject affected by the hair loss until the hair loss disorder is cured, prevented or alleviated or according to doctor's instructions.

In certain embodiments, the method comprises administration of a therapeutically effective amount of the pharmaceutical composition once or twice daily. In certain embodiments, the method comprises application of a therapeutically effective amount of the pharmaceutical composition once or twice daily.

### Definitions

As used herein a “pharmaceutical composition” refers to a preparation of one or more of the compounds described herein, or physiologically acceptable salts or solvents thereof, with other chemical components such as physiologically suitable carriers and excipients. The purpose of a pharmaceutical composition is to facilitate administration of a compound to a subject.

The term “pharmaceutically acceptable” means approved by a regulatory agency of the Federal or a state government or listed in the U. S. Pharmacopeia or other generally recognized pharmacopeia for use in animals, and more particularly in humans.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in microcapsules or formulations according to the present disclosure.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The following examples are to be considered merely as illustrative and non-limiting in nature. It will be apparent to one skilled in the art to which the present invention pertains that many modifications, permutations, and variations may be made without departing from the scope of the invention.

### EXAMPLES

### Example 1. Preparation and Stability of a 0.75% Topical Tapinarof Gel Composition

TABLE 1

| Ingredient | % in formulation |
|---|---|
| Tapinarof | 0.75 |
| DMSO (dimethylsulfoxide) | 70 |
| Propylene glycol | 25.50 |
| 2-phenoxyethanol | 0.5 |
| Methylparaben | 0.25 |
| Carbopol ®980 | 3 |

Procedure: Tapinarof was dissolved; Methylparaben was added under stirring; Carbopol® was added under stirring; 2-phenoxyethanol was dissolved in propylene glycol and added; The formulation was stirred and homogenized to obtain a homogeneous gel.

### Example 2. Preparation and Stability of a 0.5% Topical Tapinarof Gel Composition

TABLE 2

| Ingredient | % in formulation |
|---|---|
| Tapinarof | 0.5 |
| DMSO | 70 |
| Propylene glycol | 25.75 |
| 2-phenoxyethanol | 0.5 |
| Methylparaben | 0.25 |
| Carbopol ®980 | 3 |

Procedure: Tapinarof was dissolved; Methylparaben was added under stirring; Carbopol® was added under stirring; 2-phenoxyethanol was dissolved in propylene glycol and added; The formulation was stirred and homogenized to obtain a homogeneous gel.

### Example 3. Preparation and Stability of a 1% Vasoconstrictor (Such as Oxymetazoline)+1% Tapinarof Topical Gel Composition

| Ingredient | % in formulation |
|---|---|
| Oxymetazoline | 1 |
| Tapinarof | 1 |
| DMSO | 70 |
| Propylene glycol | 24.25 |
| 2-phenoxyethanol | 0.5 |
| Methylparaben | 0.25 |
| Carbopol ®980 | 3 |

Procedure: Vasoconstrictor is dissolved; Tapinarof is added under stirring; Methylparaben is added under stirring; Carbopol® is added under stirring; 2-phenoxyethanol is dissolved in propylene glycol and added. The formulation is stirred and homogenized to obtain a homogeneous gel.

### Example 4. Preparation and Stability of a 1% an EGFR Inhibitor (Such as Erlotinib)+1% Tapinarof Topical Gel Composition

| Ingredient | % in formulation |
|---|---|
| Erlotinib | 1 |
| Tapinarof | 1 |
| DMSO | 70 |
| Propylene glycol | 24.25 |
| 2-phenoxyethanol | 0.5 |
| Methylparaben | 0.25 |
| Carbopol ®980 | 3 |

Procedure: EGFR inhibitor is dissolved; Tapinarof is added under stirring; Methylparaben is added under stirring; Carbopol® is added under stirring; 2-phenoxyethanol is dissolved in propylene glycol and added. The formulation is stirred and homogenized to obtain a homogeneous gel.

### Example 5. Preparation and Stability of a 1% Vasoconstrictor (Such as Oxymetazoline)+1% EGFR Inhibitor (Such as Erlotinib)+1% Tapinarof Topical Gel Composition

| Ingredient | % in formulation |
|---|---|
| Oxymetazoline | 1 |
| Erlotinib | 1 |
| Tapinarof | 1 |
| DMSO | 70 |
| Propylene glycol | 23.25 |
| 2-phenoxyethanol | 0.5 |
| Methylparaben | 0.25 |
| Carbopol ®980 | 3 |

Procedure: Vasoconstrictor is dissolved; EGFR inhibitor is dissolved; Tapinarof is added under stirring; Methylparaben is added under stirring; Carbopol® is added under stirring; 2-phenoxyethanol is dissolved in propylene glycol and added. The formulation is stirred and homogenized to obtain a homogeneous gel.

What is claimed is:

1. A method of treating, preventing or alleviating chemotherapy-induced, radiation-therapy-induced, or hormone-replacement-therapy-induced alopecia or hair loss in a subject in need thereof, wherein the method comprises administering to the subject a therapeutically effective amount of a topical pharmaceutical composition comprising at least one aryl hydrocarbon receptor (AhR) agonist in a concentration of between 0.01% to about 5% w/w, and a pharmaceutically acceptable carrier or excipient.

2. The method of claim 1, wherein the least one aryl hydrocarbon receptor (AhR) agonist is administered in a concentration of between 0.01% to about 1% w/w, from about 1% to about 3% w/w or from about 3% to about 5% w/w.

3. The method according to claim 1, wherein the AhR agonist is selected from the group consisting of tapinarof, FICZ (6-formylindolo(3,2b)carbazole), Indirubin, ITE (2-(1′H-indole-3′carbonyl)-thiazole-4-carboxylic acid methyl ester), L-Kynurenine, a Flavonoid, Leflunomide, and Norisoboldine.

4. The method according to claim 1, wherein the AhR agonist is tapinarof.

5. The method of claim 1, wherein the composition further comprises at least one EGFR inhibitor in a concentration of from about 0.01% to about 1% w/w, from about 1% to about 3% w/w, from about 3% to about 5% w/w or from about 5% to about 10% w/w.

6. The method according to claim 4, wherein the composition further comprises at least one EGFR inhibitor in a concentration of from about 0.01% to about 1% w/w, from about 1% to about 3% w/w, from about 3% to about 5% w/w or from about 5% to about 10% w/w.

7. The method according to claim 1, wherein the composition further comprises at least one vasoconstrictor compound in a concentration of between about 0.01% to about 5% w/w.

8. The method according to claim 4, wherein the composition further comprises at least one vasoconstrictor compound in a concentration of between about 0.01% to about 5% w/w.

9. The method according to claim 5, wherein the composition further comprises at least one vasoconstrictor compound in a concentration of between about 0.01% to about 5% w/w.

10. The method according to claim 1, wherein the composition further comprises (i) at least one EGFR inhibitor in a concentration of from about 0.01% to about 1% w/w, from about 1% to about 3% w/w, from about 3% to about 5% w/w or from about 5% to about 10% w/w; and (ii) at least one vasoconstrictor compound in a concentration of between about 0.01% to about 5% w/w.

11. The method according to claim 4, wherein the composition further comprises (i) at least one EGFR inhibitor in a concentration of from about 0.01% to about 1% w/w, from about 1% to about 3% w/w, from about 3% to about 5% w/w or from about 5% to about 10% w/w; and (ii) at least one vasoconstrictor compound in a concentration of between about 0.01% to about 5% w/w.

12. The method according to claim 5, wherein the EGFR inhibitor is selected from the group consisting of erlotinib, gefitinib, lapatinib, cetuximab, panitumumab, vandetanib, necitumumab, osimertinib, and combinations thereof.

13. The method according to claim 7, wherein the vasoconstrictor compound is selected from the group consisting of oxymetazoline, xylometazoline, brimonidine, a vasopressin analog, epinephrine, norepinephrine, phenylephrine (Sudafed PE), dopamine, dobutamine, and a serotonin 5-hydroxytryptamine agonist (a triptan).

14. The method according to claim 1, wherein the topical pharmaceutical composition is formulated as a cream, an ointment, a gel, a lotion, a spray, a shampoo, a patch, or a foam.

15. The method according to claim 5, wherein the topical pharmaceutical composition is formulated as a cream, an ointment, a gel, a lotion, a spray, a shampoo, a patch, or a foam.

16. The method according to claim 7, wherein the topical pharmaceutical composition is formulated as a cream, an ointment, a gel, a lotion, a spray, a shampoo, a patch, or a foam.

17. The method according to claim 10, wherein the topical pharmaceutical composition is formulated as a cream, an ointment, a gel, a lotion, a spray, a shampoo, a patch, or a foam.

18. The method according to claim 1, wherein the topical pharmaceutical composition is administered before, during and/or after the chemotherapy, radiotherapy or hormone-replacement protocol.

19. The method according to claim 5, wherein the topical pharmaceutical composition is administered before, during and/or after the chemotherapy, radiotherapy or hormone-replacement protocol.

20. The method according to claim 7, wherein the topical pharmaceutical composition is administered before, during and/or after the chemotherapy, radiotherapy or hormone-replacement protocol.

21. The method according to claim 10 wherein the topical pharmaceutical composition is administered before, during and/or after the chemotherapy, radiotherapy or hormone-replacement protocol.

22. The method according to claim 5, wherein the at least one AhR agonist and the at least one EGFR inhibitor exhibit an additive or synergistic effect.

23. The method according to claim 7, wherein the at least one AhR agonist and the at least one vasoconstrictor compound exhibit an additive or synergistic effect.

24. The method according to claim 10, wherein the at least one AhR agonist, the at least one EGFR inhibitor and the at least one vasoconstrictor compound exhibit an additive or synergistic effect.

25. The method according to claim 1, wherein the alopecia is selected from the group consisting of alopecia areata totalis, alopecia areata universalis and androgenetic alopecia, telogen effluvium, tinea capitis, hypotrichosis, and hereditary hypotrichosis simplex.

26. The method according to claim 1, wherein the method comprises topical application of a therapeutically effective amount of the topical pharmaceutical composition to the scalp or skin portion of the subject affected by the hair loss until the hair loss disorder is cured, prevented or alleviated or according to doctor's instructions.

27. The method according to claim 1, wherein the method comprises application of a therapeutically effective amount of the topical pharmaceutical composition once or twice daily.

28. The method according to claim 5, wherein the EGFR inhibitor is erlotinib.

29. The method according to claim 7, wherein the vasoconstrictor is oxymetazoline.

30. The method of claim 10, wherein the EGFR inhibitor is erlotinib and the vasoconstrictor is oxymetazoline.
